(19) United States
(12) Patent Application Publication
(10) Pub. No.: US 2025/0258065 A1
(43) Pub. Date: Aug. 14, 2025

(54) Sampling device for liquid foams

(71) Applicant: Board of Trustees of Western Michigan University, Kalamazoo, MI (US)

(72) Inventors: Ethan Samuel Coffin, Ann Arbor, MI (US); Donald M. Reeves, Kalamazoo, MI (US)

(21) Appl. No.: 19/054,538

(22) Filed: Feb. 14, 2025

Related U.S. Application Data
(60) Provisional application No. 63/553,198, filed on Feb. 14, 2024.

(51) Int. Cl. G01N 1/12

(52) U.S. Cl. CPC G01N 1/12

(57) ABSTRACT

Aspects and embodiments of the present disclosure may include a device for sampling wet foams including an inner sampling tube, which includes a top plate and a bottom plate forming an inner volume, and one or more openings in the sampling tube wall capable of allowing wet foam from the outside environment to enter the inner volume, where the junction of the bottom plate and inner sampling tube is watertight; and an outer adjustable tube, where the outer adjustable tube is capable of sliding over the inner sampling tube, such that the one or more openings may be exposed or closed to the environment by the outer adjustable tube, allowing a wet foam sample to be held in the inner volume, as well as methods of using the same.

### CROSS-REFERENCE TO RELATED APPLICATIONS

The present application claims the benefit of U.S. Provisional No. 63/553,198, filed Feb. 14, 2024, the contents of which are incorporate herein in their entirety.

### BACKGROUND OF THE INVENTION

Aspects and embodiments of the present disclosure may include a novel sampling device to conserve foam volume during collection, allowing spatial and temporal heterogeneity in foam composition to be accurately and more easily determined. It is very difficult to collect representative foam samples as they readily collapse, especially dynamic wet foams like those forming at waste-water treatment plants (WWTP) and others. Previous studies have used a variety of foam collection methods, including hand scooping samples into bottles (Schaefer et al., 2022; Smith et al., 2023a) or using screened nets when difficult to access (AECOM, 2021). These methods have limited reproducibility, subject the foam to drainage during sample retrieval, and do not conserve foam volume, which is necessary to determine liquid content. Therefore, a specific wet foam sampling device was developed to collect consistent and comparable samples while conserving foam volume.

### BRIEF SUMMARY OF THE INVENTION

Some embodiments of present disclosure may include a device for sampling wet foams including: an inner sampling tube, which includes a top plate and a bottom plate forming an inner volume, and one or more openings in the sampling tube wall capable of allowing wet foam from the outside environment to enter the inner volume, where the junction of the bottom plate and inner sampling tube is watertight; and an outer adjustable tube, where the outer adjustable tube is capable of sliding over the inner sampling tube, such that the one or more openings may be exposed or closed to the environment by the outer adjustable tube, allowing a wet foam sample to be held in the inner volume.

In others, the junction of the top plate and inner sampling tube is watertight. In yet others, the junctions of the top plate and bottom plate are each made watertight with one or more O-rings.

In other embodiments, at least a portion of the material of the top plate is transparent, and/or at least a portion of the material of the bottom plate is transparent.

In other embodiments, the device further includes a handle allowing the device to be deployed at a distance from a user of the device, where the handle is attached to the lower sampling tube. In others, the handle includes an extendable pole to adjust the distance between the user and the device. In others, the handle further includes a spacer attached to the lower sampling tube at one end and the pole at the other end.

In other embodiments, the device further includes a pull attached to the outer adjustable tube, allowing the outer adjustable tube to be raised and lowered at some distance from a user. In others, the pull is a rope, attached at least one point to the outer adjustable tube.

Some aspects of the present disclosure may include methods for sampling wet foams including, deploying a sampler device into the foam, the sampler device including: an inner sampling tube, an inner sampling tube, which includes a top plate and a bottom plate forming an inner volume, and one or more openings in the sampling tube wall capable of allowing wet foam from the outside environment to enter the inner volume, where the junction of the bottom plate and inner sampling tube is watertight; and an outer adjustable tube, where the outer adjustable tube is capable of sliding over the inner sampling tube, such that the one or more openings may be exposed or closed to the environment by the outer adjustable tube, allowing a wet foam sample to be held in the inner volume.

In others, the junction of the top plate and inner sampling tube is watertight. In yet others, the junctions of the top plate and bottom plate are each made watertight with one or more O-rings.

In other aspects, at least a portion of the material of the top plate is transparent, and/or at least a portion of the material of the bottom plate is transparent.

In other aspects, the device further includes a handle allowing the device to be deployed at a distance from a user of the device, where the handle is attached to the lower sampling tube. In others, the handle includes an extendable pole to adjust the distance between the user and the device. In others, the handle further includes a spacer attached to the lower sampling tube at one end and the pole at the other end.

In other aspects, the device further includes a pull attached to the outer adjustable tube, allowing the outer adjustable tube to be raised and lowered at some distance from a user. In others, the pull is a rope, attached at least one point to the outer adjustable tube.

### BRIEF DESCRIPTION OF THE DRAWINGS

The present invention(s) are illustrated by way of example and not limitation with reference to the accompanying drawings, in which like references generally indicate similar elements or features.

FIG. 1 depicts an example embodiment sampler of the present disclosure in the closed position;

FIG. 2 depicts an example embodiment sampler of the present disclosure in the open position;

FIG. 3 depicts a top view an outer adjustable tube of an example embodiment of the present disclosure;

FIG. 4 depicts two diagrammatic longitudinal sections (A, B) of an inner sampling tube of an example embodiment of the present disclosure;

FIG. 5 shows an example embodiment of the present disclosure with a pole and rope attached to the body of an example sampler;

FIG. 6 is a diagrammatical longitudinal section of an example embodiment of the sampler;

FIG. 7 is a photograph of an embodiment sampler of the present disclosure being used to sample foam from a post-tertiary cascade at a wastewater treatment plant (WWTP);

FIG. 8 is a graph showing the estimated surface tension vs. liquid content of 37 samples of WWTP foam and effluent (as a control) from various locations in post-tertiary foam;

FIG. 9 is a graph showing the ability to accurately determine the concentration of PFAS contamination in WWTP foams.

### DETAILED DESCRIPTION

As noted above, it is very difficult to collect representative foam samples as they readily collapse, especially dynamic wet foams. Methods of the prior art have limited reproducibility, subject the foam to drainage during sample retrieval, and do not conserve foam volume, which is necessary to determine liquid content.

Sampling devices of the present disclosure are designed such that, when open, foam is free to flow into and fill the sampler before the lid is dropped, shearing the foam and isolating the sample inside. The sampling device may be mounted, in some embodiments, on fixed or extendable grab sampling rods and the like to collect samples at some distance from the user.

The sampler of the present disclosure is most appropriate for foams wet enough to flow into and occupy the entire volume of the sampler. It is designed to minimize potential under-sampling, when foam does not completely fill the sampler, by allowing it to flow in radially with visual checks via the clear top to ensure the foam reaches the center of the sampler. Over-sampling, when the foam collapses and additional foam can flow into the sampler, may be mitigated by limiting the time the lid is open to a few seconds.

Foams that may be sampled using embodiments and aspects of the present disclosure may have a liquid content of about 1.5% and greater, 2% and greater, between 2% and 20%, 2% and 10%, 2% and 7%, 2% and 6.5%.

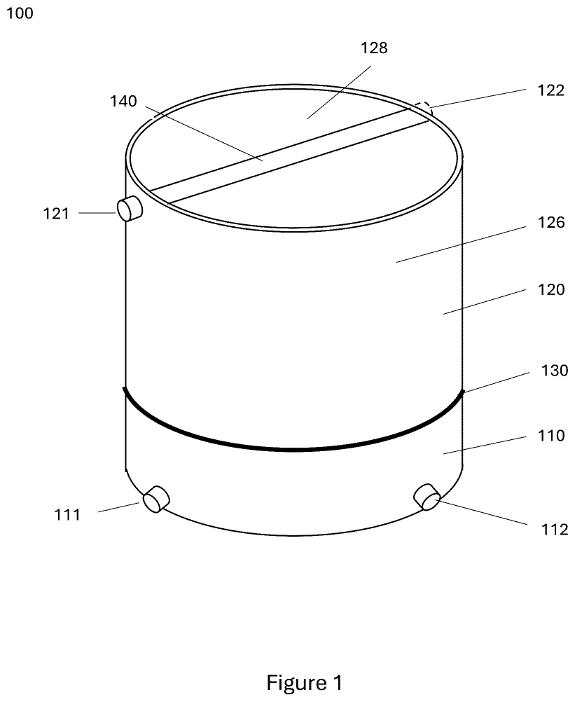

FIG. 1 depicts an example embodiment sampler 100 of the present disclosure in the closed position. The sampler 100 here includes an inner sampling tube 110 and an outer adjustable tube 120, which are configured such that outer adjustable tube 120 has a slightly larger inner diameter than the outer diameter of inner tube 110 and may be slid up and down 110 maintaining a relatively watertight proximity between the tubes.

In some embodiments, outer tube 120 has an outer surface 126 and inner surface 128, the latter of which slides over the outer surface of inner tube 110. A hydrophobic substance such as wax may be applied to all or a portion of 128 and/or the outer surface of 110 to aid in water resistance and/or slideability but is not usually necessary due to the relative rapidity in which most foam samples should be taken as they may be are dynamic systems.

Tube 110 and tube 120 may be made from any suitable material, such as metal or plastic, but should be relatively rugged and cleanable for multiple use and to avoid cross-contamination of samples. In some embodiments, a relatively light metal non-reactive metal such as aluminum may be used, in others plastics, such as PVC may be used, or a combination thereof (e.g., 110 made of a different material than 120).

Referring to FIG. 1, a rubber O ring 130 is used to stop the movement of 120 and/or seal the junction between the end of 120 and the surface of 110. One or more lower ports 111 and 112 (one or more may be present on the far side of inner tube 110 (not shown)) may be present on 110. These may serve as attachment points for various means of holding 100 while in use, and/or channels to accept screws or other attachment means known in the art for interior partitions of 110, described more fully below.

Brace 140 may be present that extends through outer tube 120 and having brace ends 121 and 122 to hold brace 140 and/or provide attachment points for a user to lower and raise outer tube 120, described more fully below.

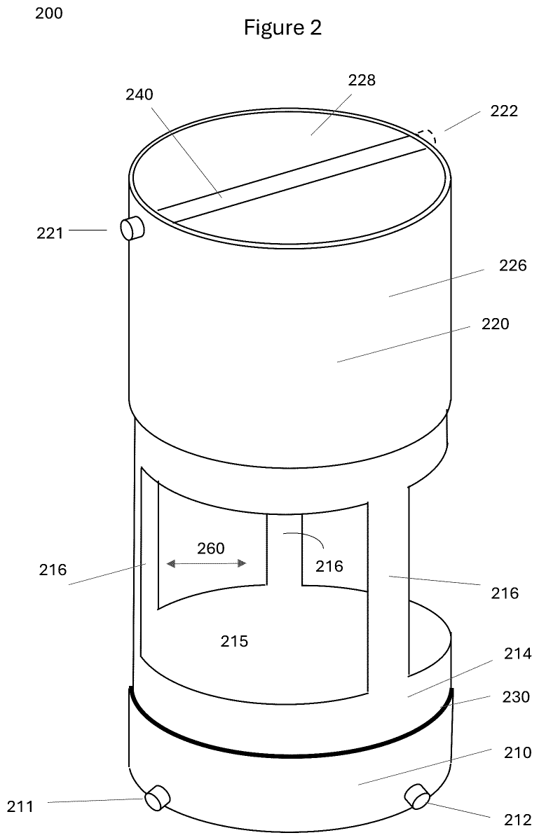

FIG. 2 depicts an example embodiment sampler 200 of the present disclosure in the open position. The sampler 200 includes an inner sampling tube 210, with ports 211 and 212, and an outer adjustable tube 220; outer adjustable tube 220 has been slid up inner tube 210. Outer tube 220 has an outer surface 226 and inner surface 228, the latter of which slides over the outer surface 214 of inner tube 210.

Outer adjustable tube 220 may be slid from the location of rubber O ring 230. Control of the upper range of movement of 220 is discussed more fully below. Brace 240 and/or brace ends 221 and 222 may be used, in some embodiments, as attachment points to raise and lower 220.

Inner sampling tube 210 has one or more openings 260, with supports 216 between or delineating each opening 260 that allow foam to flow into the inner volume 215 of sampling tube 260. Openings 260 should be large enough to allow foam to flow freely into the sampler, such that, in most embodiments, supports 216 should be narrow enough to allow free flow while still structurally supporting the device. Openings 260 may be of any suitable shape, such a rectangular or circular, or oval; in some applications will be symmetrically disposed about inner tube 210, but may be asymmetrical in free-flowing foams, for instance, to collect foam moving in one direction.

Inner sampling tube 210 and openings 260 may be of any desired height, based on the size of a foam layer in a particular application or a particular portion of the foam layer desired to be sampled. In some applications, opening 260 will be the same size, larger than or smaller than the foam layer. Outer tube 220 may be of any height as long as it can cover openings 260 when in the closed position (see FIG. 1).

Similarly, sampler 200 (and thus diameters of 210 and 220) may be of any desired overall diameter. Considerations of sample size needed, foam density and flowability, overall weight and ease of use may lead to a choice of a smaller or larger diameter sampler 210.

Inner 210 and outer 220 tubes are shown with a circular cross section, but any shaped cross section may be used (oval, square, circular with a flat edge to sample basin sides, for example) as long as the tubes can slide over each other with a relatively water-tight seal.

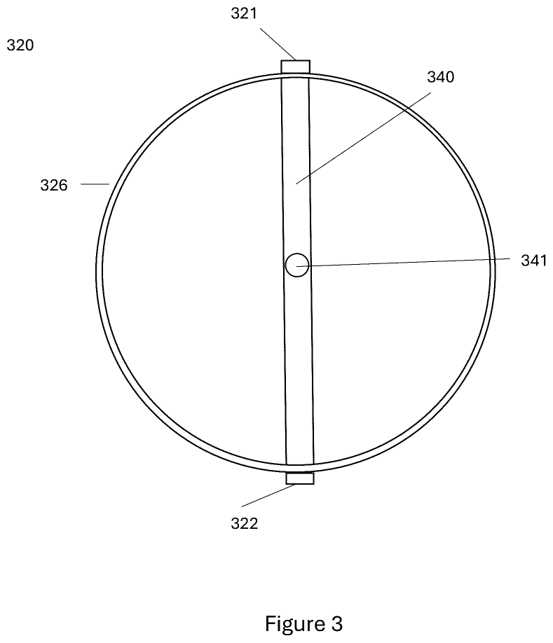

FIG. 3 depicts a top view of an example embodiment of an outer adjustable tube 320, with outer surface 326. Brace 340 and/or brace ends 321 and 322 may be used, in some embodiments, as attachment points for ropes, poles, handles, etc. to raise and lower 320. Further, brace 340 may contain one or more openings 341. Openings may be used for attachment points to raise and lower 320. In some embodiments, opening 341 may be used to allow passage of a stop rod, discussed below, to limit the upward movement of 320.

In some embodiments, outer tube 320 may contain ends 321 and 322, or one or more ports similar to lower ports 211 and 212 on FIG. 2. A brace may be present or absent or may be substituted with a covering over all or part of the top of outer tube 320. Braces may be various thicknesses and widths.

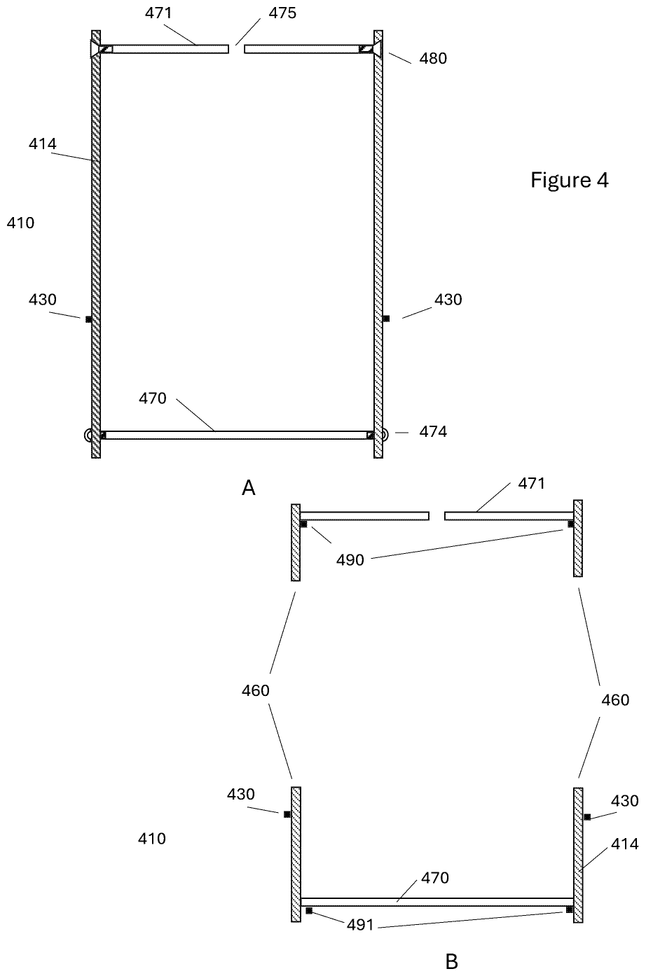

FIG. 4 depicts two diagrammatic longitudinal sections of an inner sampling tube 410 of an example embodiment of the present disclosure, to show certain features of the embodiments. FIG. 4A shows 410 with tube wall 414. O-ring 430 may be present on the outer surface of 414. Foam is held in the sampler by two end plates top place 471 and bottom plate 470. In some embodiments, one or more the end plates are made of clear acrylic or other transparent material to allow monitoring of foam intake and placement within the foam layer, for instance, to determine when the sampler has touched the foam layer, is within the foam layer, or has reached an underlying liquid layer beneath the foam, if present. Any other suitable material may be used, whether opaque, semi- or fully transparent.

Top 471 and bottom 470 plates are held in place with one or more fasteners 480 and 474, respectively, through the wall 414 into the plates to attach the plates to the tube wall 414. Notably, fastener 480 is recessed in wall 414 to allow the outer tube to slide freely. Other suitable attachment means, such as welds, adhesives, caulks, O-rings (see FIG. 4B) insertion into grooves in the inner tube 410, tabs, ridges, and other attachment means known to persons of skill in the art may be used, but care must be taken for a secure and water-tight fit for accurate, repeatable volumes and liquid measurements. One or more holes 475 may be present in top plate 471 to allow insertion of a stop rod (see FIG. 4B), pressure equalization to aid sample intake or removal, and the like.

FIG. 4B shows a different diagrammatical longitudinal section showing openings 460. Further, two additional O-rings 490 and 491 place below top 471 and bottom place 470, respectively, to aid in watertightness and hold the plates securely. Thes may be placed below, between the plates and wall 414 and/or above one or more of the plates in some embodiments.

In some embodiments, the sampler also includes a handle, allowing the device to be securely held and deployed at some distance from the user. In some embodiments, the handle may be a long pole or rope, etc. attached to the inner sampling tube. In others, the pole or rope may be extendable. In others, the body of the sampler may be attached to a horizontal spacer; the rope or pole is attached at the other end of the spacer to avoid the pole or rope interfering with foam flow into the inner chamber.

In some embodiments, the sampler also includes a pull attached to the outer adjustable tube, allowing, at some distance from the user, the outer adjustable tube to be pulled or moved up and down to cover or uncover the openings into the inner volume located in the inner sampling tube. In some embodiments, the pull may be a long pole or rope, etc. attached to the outer sampling tube at one or more points of attachment. In others, the pull may be attached to the brace or covering of the outer adjustable tube.

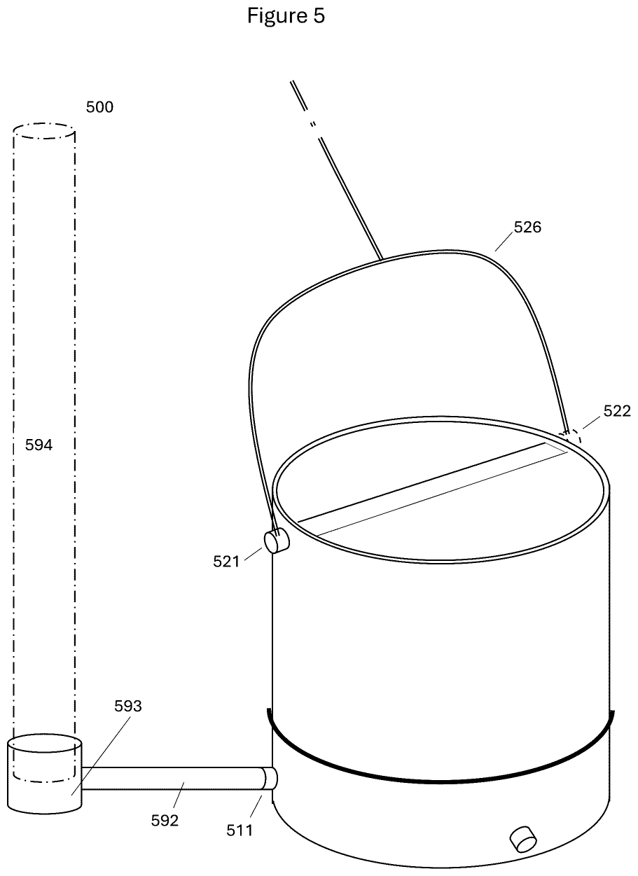

FIG. 5 shows an example attachment of pole 594 and rope 526 to sampler 500, allowing for operation of the sampler at a distance from the user. In general, pole 594 should be attached to inner sampler tube 510, allowing secure and precise placement of the sampler, and rope 526 should be attached to outer sampler tube to allow it to be moved up and down.

Pole 594 in most embodiments is relatively rigid to facilitate the sampler remaining upright, although a rope, pulley system or other adjustable means of raising and lowering the sampler 500 is possible as well. In some embodiments, pole 594 is capable of having an adjustable length (i.e. a “telescoping pole”). In the embodiment shown, pole 594 is held by receptacle 593. Pole 594 may be capable of being screwed into receptacle 593, such that a standard off-the shelf telescoping poles may be used. Receptacle 593 may attach to spacer 592, which attaches into port 511. Sufficient space between the pole and sampler 500 body may be needed to avoid foam disturbance near the sampler. Other means and points of attachment are possible, such as running through the center or other part of the interior of the sampler and attaching to the bottom or sides of the inner sampler tube 510; care should be taken to ensure slideability of upper tube 520 and watertightness of sampling tube 510.

In some embodiments, rope 526 may be used to raise and lower the upper sampler 520. Other means, such as rope or sliding rod, which may slide in or along pole 594 in whole or in part may also be used, as well as any suitable means and material that can securely attach to upper tube 520. Rope 526 may divide (or form a loop, or two ropes and a loop attached to a central rope 526 and attach to ends 521 and 522; alternatively, two or more ropes can be used to raise and lower 520. An advantage to multiple points of attachment at the lateral edge is that 520 can raise and lower evenly. As is well known in the art, this may also be accomplished with one point of attachment at various locations on 520 by a stiffer rope 526, rod or other means of remotely lowering and raising the outer tube 520.

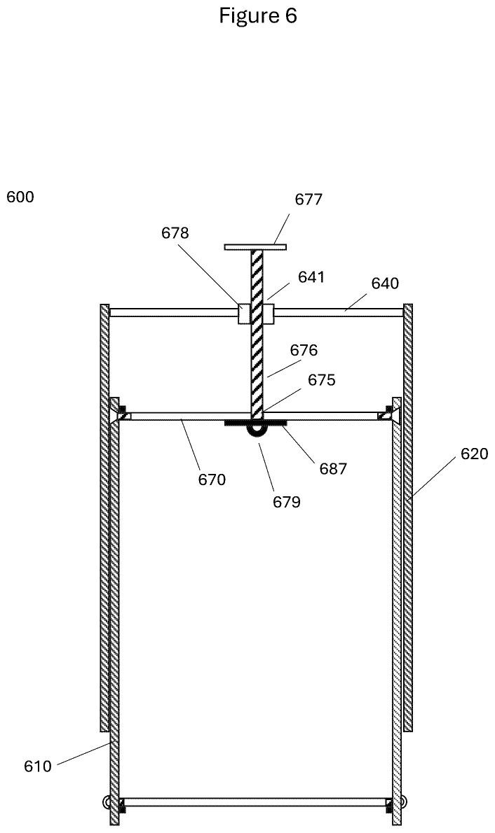

FIG. 6 is a diagrammatical longitudinal section of an example embodiment of sampler 600, showing a stop rod 676 to limit upper movement of upper tube 620 as it slides over inner tube 610. Stop rod 676 has a bolt head 679 and a stop 677 and is placed through hole 675 in upper plate 670 of inner tube 610. A washer 687, stops leakage of the contents of inner tube 610 through hole 675. Gasket 678 fitting around hole 641 allows stop rod 676 to extend through hole 641 in brace 640, and outer tube 620 to slide along stop rod 676, until gasket 678 hits 677, stopping movement. Other means of checking the movement of outer tube 620 known to persons of skill in the art are also possible.

Example 1. An example embodiment was constructed, an aluminum vessel with an internal volume of 410 ml, manufactured from an 8.9 cm diameter aluminum tube. Three 4.6 cm tall rectangular openings were cut out to allow foam to flow radially in, leaving three thin sections for support. The total area of the three openings was 75 cm². The top and bottom of the tube were covered with transparent acrylic and sealed with rubber O-rings. An additional section of aluminum tubing of slightly larger diameter was mounted to the top of the sampler and serves as a lid that can be opened and shut vertically with a cord.

Example 2. The sampler embodiment detailed in the above example was used to obtain foam samples from the Kalamazoo Wastewater Reclamation Facility (KWRF) located in Kalamazoo, MI. The KWRF receives an average of 95 million L/day of combined industrial (20%) and municipal (80%) wastewater which it treats with primary screens and sedimentation, secondary activated sludge with addition of powdered activated carbon and biological nutrient reduction, followed by tertiary chlorination and sand filtration (Vitale et al., 2023). Since 2018, KWRF has participated in the industrial pretreatment program initiative of Michigan Department of Environment, Great Lakes and Energy (EGLE), to identify and reduce influent sources of per- and polyfluorinated alkyl substances (PFAS).

PFAS are a family of 10,000+ compounds containing at least one carbon-fluorine bond, widely used in manufacturing, commercial, and consumer products (Buck et al., 2021). PFAS bioaccumulate in plants, animals, and humans, causing adverse reproductive and developmental outcomes, altered immune response, and cancer at very low concentrations (Fenton et al., 2021). Due to their resistance to degradation and the lack of efficient destructive technologies, PFAS cycle throughout engineered systems until ultimately being released to the natural environment (Stoiber et al., 2020). Wastewater treatment plants (WWTP) passively receive PFAS from landfill leachate, municipal and industrial sources, and have been identified as key environmental discharge points (Cookson and Detwiler, 2022; Helmer et al., 2022; Lang et al., 2017).

Foam fractionation (FF) is receiving increasing attention as a simple, efficient, and scalable technology to concentrate and remove PFAS from water. FF takes advantage of the surface-active properties of PFAS, owing to competition between the hydrophobic fluorinated tail and hydrophilic functional head group (Kancharla et al., 2022; Wang et al., 2023; Ziaee et al., 2021) PFAS removal via FF is a complex process dependent on many parameters such as the co-surfactants present (e.g., biopolymers from microorganisms), operational conditions of the system, and ionic activity (Buckley et al., 2023; Merz et al., 2011; Vo et al., 2023).

One potential method of avoiding costly FF methods, is to investigate PFAS removal in foams that naturally form at WWTP. Foaming in WWTP (especially in secondary aeration tanks) is very common, stimulated by the production of bio-surfactants from filamentous bacteria, and the presence of manufactured surfactants (Collivignarelli et al., 2020; Palmer and Hatley, 2018). Recently, Schaefer et al. (2023) and Smith et al. (2023a) found the enrichment of individual PFAS compounds (e.g., the increase in PFAS concentrations in the foam compared to the wastewater) across these 12 plants varied by orders of magnitude and was highly sensitive to plant-specific parameters and operation. Despite this variability, foams generally followed the expected trend of greater enrichment as C-F chain length increased. Long chained PFAS showed concentrations in foam up to 105 times greater than in the host wastewater.

While these results are promising for using foams to remove PFAS from wastewater, most WWTP would need to be modified to generate enough foam for meaningful PFAS mass removal (Smith et al., 2023a), as the efficiency of FF is inversely related to the scale of the system (Kown, 1971).

Cascades can generate foam by entrapping air during the shearing between the plunging cascade and the receiving pool (Kister et al., 2023; McKeogh and Ervine, 1981). WWTP often use cascades to aerate wastewater and regulate outfall elevation, and these systems often generate foam as an unintended byproduct. Although the previously mentioned two studies have identified PFAS in WWTP foams, this is the first study determining PFAS concentrations and enrichment in foams forming at WWTP cascades.

Use of an embodiment of a foam sampling device of the present disclosure enabled the quantification of liquid content for 36 foam samples collected from 3 distinct regions at a WWTP, providing unique insights into mechanisms controlling PFAS enrichment and the feasibility of cascade driven FF for PFAS removal at the WWTP scale.

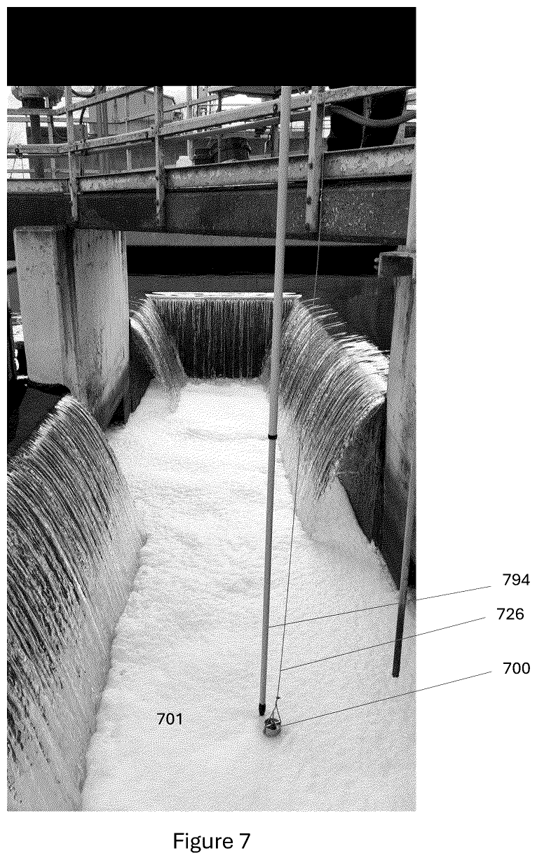

FIG. 7 is a photograph of an embodiment sampler 700 of the present disclosure being used to sample foam from a post-tertiary cascade. Tertiary wastewater has completed treatment and is being discharged back into a local water source (here, the Kalamazoo River). Just prior to discharge, foam is held (i.e. river foam) until it partially or completely dissolves to avoid surface foam visible on the river. The cascade is primarily used to prevent backflow of the river Into the treatment plant. To effectuate contact with the foam layer 701 and operate the sampler pole 794 and rope 726 are attached to the outer and inner tubes as described more fully above.

The consistent volume, ability to take a representative sample, and rapid acquisition of samples allows sensitive determination of foam properties, after being sampled and then subsequently analyzed. The sampler was decontaminated between samples by rinsing with DI water, 100% methanol, and a second DI rinse.

For instance, surface tension of effluent and collapsed foam collected from all eight-days were measured using the Wilhelmy plate method with a CBVP-Z surface tensiometer (Kyowa Interface Science Ltd, Japan). Due to the dynamic nature of surfactants, multiple measurements were taken over the course of 15 minutes, ensuring a fresh initial surface by aliquoting immediately before taking the initial measurement. Values reported for a surface age of 15 minutes were used for equilibrium surface tensions.

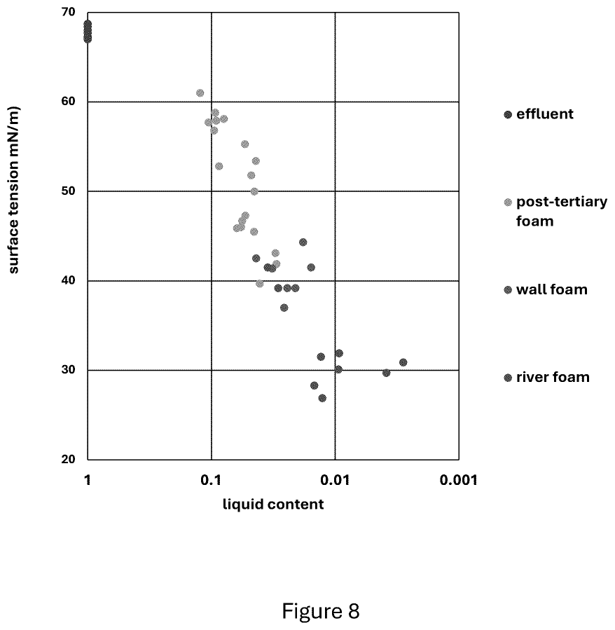

FIG. 8 shows the estimated surface tension vs. liquid content of 37 samples of WWTP foam and effluent (as a control) from various locations in post-tertiary foam. Wall foam is foam just behind the post-tertiary cascade in a less-turbulent area; as noted above, river foam is foam just prior to discharge in the river. Note that effluent was sampled directly with a bucket, and the river foam was too dry to use the sampler but could be sampled using a polyethylene bucket. The river foams were the driest (0.283-1.47%), followed by the wall foams (2.10-4.36%), and then the wet post-tertiary foams (2.98-6.24%).

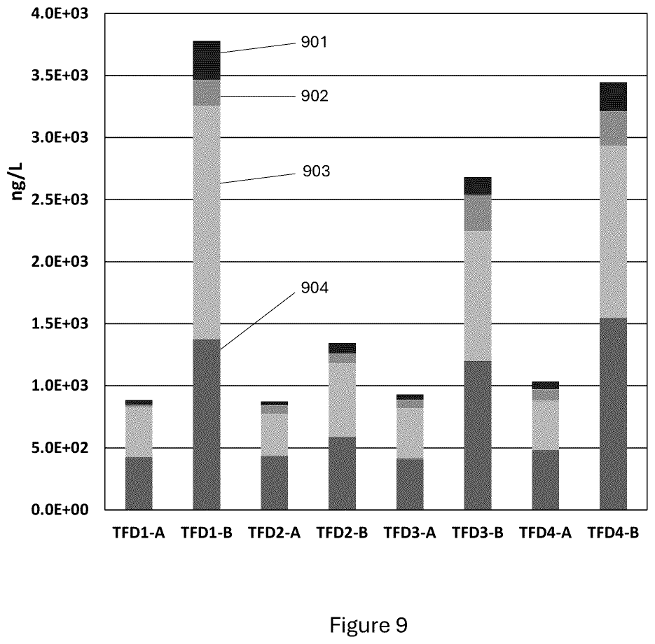

Effluent and foam sample volumes were divided into two HDPE bottles (1-L or 125-ml), one of which was sent to an external Michigan PFAS-certified laboratory for quantification of 40 PFAS compounds via liquid chromatography with tandem mass spectrometry (LC/MS/MS) following the EPA draft 1633 method (EPA, 2023). FIG. 9 shows the ability to accurately characterize and thus compare the concentrations of various types of PFAS contaminants in a representative, tertiary cascade location: PFSA precursors (shade labeled as 901); PFCA precursors (902); PFSA (903), and PFCA (904).

In summary, foam sampler described herein may have applications for foam sampling in any small- or large-scale processes. Foam separation has many industrial applications such as protein and enzyme separation in the food and pharmaceutical industries (Burghoff, 2012), metal extraction in mining (Rangarajan & Sen, 2013), as well as dye recovery in the textile industry (Lu et al., 2010), and thus the sampler can aid in precisely characterizing the foam content resulting from industrial processes in time and space.

Persons of skill in the art will realize that the above examples detail example embodiments and aspects and are provided to enable any person skilled in the art to practice the disclosure. The various modifications to these embodiments will be readily apparent to those skilled in the art, and the generic principles defined herein may be applied to other embodiments without the use of inventive faculty. Thus, the present disclosure is not intended to be limited to the embodiments shown herein but is to be accorded the widest scope consistent with the principles and novel features disclosed herein.

What is claimed is:

1. A device for sampling wet foams comprising,
an inner sampling tube, comprising
a top plate and a bottom plate forming an inner volume, and
one or more openings in the sampling tube wall capable of allowing wet foam from the outside environment to enter the inner volume, wherein the junction of the bottom plate and inner sampling tube is watertight; and
an outer adjustable tube,
wherein the outer adjustable tube is capable of sliding over the inner sampling tube, such that the one or more openings may be exposed or closed to the environment by the outer adjustable tube, allowing a wet foam sample to be held in the inner volume.

2. The device of claim 1, wherein the junction of the top plate and inner sampling tube is watertight.

3. The device of claim 2, wherein the junctions of the top plate and bottom plate are each made watertight with one or more O-rings.

4. The device of claim 1, wherein at least a portion of the material of the top plate is transparent.

5. The device of claim 1, wherein at least a portion of the material of the bottom plate is transparent.

6. The device of claim 1, further comprising
a handle allowing the device to be deployed at a distance from a user of the device, wherein the handle is attached to the lower sampling tube.

7. The device of claim 6, wherein the handle comprises an extendable pole to adjust the distance between the user and the device.

8. The device of claim 6, wherein the handle further comprises
a spacer, wherein the spacer is attached to the lower sampling tube at one end and the pole at the other end.

9. The device of claim 1, further comprising a pull attached to the outer adjustable tube, allowing the outer adjustable tube to be raised and lowered at some distance from a user.

10. The device of claim 9, wherein the pull is a rope, attached at least one point to the outer adjustable tube.

11. A method for sampling wet foams comprising,
deploying a sampler device into the foam, the sampler device comprising:
an inner sampling tube, comprising
a top plate and a bottom plate forming an inner volume, and
one or more openings in the sampling tube wall capable of allowing wet foam from the outside environment to enter the inner volume, wherein the junction of the bottom plate and inner sampling tube is watertight; and
an outer adjustable tube,
wherein the outer adjustable tube is capable of sliding over the inner sampling tube, such that the one or more openings may be exposed or closed to the environment by the outer adjustable tube, allowing a wet foam sample to be held in the inner volume;
raising the outer adjustable tube to expose the one or more openings and allowing foam to enter the inner volume; and
lowering the outer adjustable tube to close the one or more openings to obtain the foam sample.

12. The method of claim 11, wherein the junction of the top plate and inner sampling tube is watertight.

13. The method of claim 12, wherein the junctions of the top plate and bottom plate are each made watertight with one or more O-rings.

14. The method of claim 11, wherein at least a portion of the material of the top plate is transparent.

15. The method of claim 11, wherein at least a portion of the material of the bottom plate is transparent.

16. The method of claim 11, further comprising
a handle allowing the device to be deployed at a distance from a user of the device, wherein the handle is attached to the lower sampling tube.

17. The method of claim 16, wherein the handle comprises an extendable pole to adjust the distance between the user and the device.

18. The method of claim 16, wherein the handle further comprises
a spacer, wherein the spacer is attached to the lower sampling tube at one end and the pole at the other end.

19. The method of claim 11, further comprising a pull attached to the outer adjustable tube, allowing the outer adjustable tube to be raised and lowered at some distance from a user.

20. The method of claim 19, wherein the pull is a rope, attached at least one point to the outer adjustable tube.
